(19) United States
(12) Patent Application Publication
(10) Pub. No.: US 2025/0305007 A1
(43) Pub. Date: Oct. 2, 2025

(54) METHODS AND HOST CELLS USEFUL FOR PRODUCTION OF MEVALONATE FROM SYNGAS

(71) Applicant: THE REGENTS OF THE UNIVERSITY OF CALIFORNIA, Oakland, CA (US)

(72) Inventors: Justin PANICH, Berkeley, CA (US); Sara TEJEDOR SANZ, Oakland, CA (US); Neha BANSAL, Berkeley, CA (US); Eric SUNDSTROM, San Mateo, CA (US)

(21) Appl. No.: 19/170,014

(22) Filed: Apr. 3, 2025

Related U.S. Application Data
(63) Continuation-in-part of application No. 18/825,984, filed on Sep. 5, 2024.
(60) Provisional application No. 63/569,830, filed on Mar. 26, 2024.
(60) Provisional application No. 63/638,578, filed on Apr. 25, 2024.

(51) Int. Cl. C12P 7/42; C12N 1/20; C12N 9/04; C12N 9/10; C12P 7/26; C12R 1/01

(52) U.S. Cl. CPC C12P 7/42; C12N 1/20; C12N 9/0006; C12N 9/1029; C12P 7/26; C12Y 101/03004; C12Y 203/01009; C12Y 203/0301; C12R 2001/01

(57) ABSTRACT

The present invention provides for a method or system comprising using a genetically modified host to convert syngas and/or a mixture of CO₂/H₂ to mevalonate, a precursor for a sesquiterpene or sustainable aviation fuel.

### CROSS-REFERENCE TO RELATED APPLICATIONS

This application claims the priority benefit of U.S. Provisional Patent Application Ser. No. 63/638,578, filed Apr. 25, 2024, and claims the priority benefit as a continuation-in-part application of U.S. patent application Ser. No. 18/825,984, filed Sep. 5, 2024, which claims priority benefit of U.S. Provisional Patent Application Ser. No. 63/569,830, filed Mar. 26, 2024, which are hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of producing mevalonate.

### BACKGROUND OF THE INVENTION

Gas fermentations are inexpensive and selective methods to fix carbon into desired products if reducing equivalents in the form of electrons are supplied. However, gas fermentations are constrained by the low solubility of gases in liquids and the inefficient delivery of electrons to microorganisms in bioreactors. Electricity has proven to effectively provide the energy source needed by microorganisms directly or via intermediates that are produced electrocatalytically (i.e., formate, H₂). However, current approaches deliver electrons using flat and/or static electrodes, which poses (bio) catalytic and electron transfer limitations related to poor mass transfer and low electrocatalytic surface area. Thus, these processes don't achieve high enough production rates and energy conversion efficiencies to be scalable.

Previous studies have explored electrochemical processes to transform CO₂ into chemical products, but achieving high efficiency has proven challenging. In addition, issues related to the electroconductive materials and electrode geometries significantly hinder the technology's expansion to pilot scale.

As the climate crisis continues, sustainable fuel and chemical solutions are needed. Reimagining the chemical industry and replacing petroleum-based products with those that are biomanufactured promises to mitigate several gigatons of greenhouse gas (GHG) emissions per year, decelerating climate change. A promising route to sustainable chemical production is directly using GHGs as feedstocks for chemical production, either through electrolysis, biomanufacturing, or hybrid solutions that integrate aspects of both. To this end, several anaerobic and aerobic microbial chassis have been developed to convert C₁ GHGs (CO₂, CO, CH₄) to value-added products, including Clostridium autoethanogum, Cupriavidus necator H16, and Methylotuvimicrobium alcaliphilum (Heffernan et al. 2020; Panich et al. 2021; Panich et al. 2024; Calvey et al. 2023; Goswami et al. 2024).

### SUMMARY OF THE INVENTION

The present invention provides for a host cell to convert syngas (i.e., mixtures of CO/CO₂/H₂/O₂) or mixtures of CO₂/H₂ to mevalonate (MVL).

The present invention provides for a genetically modified host cell (such as Hydrogenophaga pseudoflava) capable of converting syngas (such as a mixture of CO/CO₂/H₂/O₂) or a mixture of CO₂/H₂ to mevalonate (MVL). MVL is a precursor for biofuel, such as a sesquiterpene or sustainable aviation fuel (SAF).

In some embodiments, the genetically modified host cell is a strain capable of expressing a three-gene construct, comprising acetoacetyl-CoA thiolase (atoB) (such as E. coli AtoB), hydroxymethylglutaryl-CoA synthase (HMGS) (such as S. cerevisiae HMGS, such as S. cerevisiae ERG13), and hydroxymethylglutaryl-CoA reductase (HMGR) (such as Delftia acidovorans HMGR or S. cerevisiae HMG1), or any polypeptide having a corresponding enzymatic activity and an amino acid sequence substantially identically to the amino acid sequence of a corresponding wild type enzyme. In some embodiments, the genetically modified host cell comprises an increased expression of one or more of AtoB, hydroxymethylglutaryl-CoA synthase (HMGS), hydroxymethylglutaryl-CoA reductase (HMGR), and/or mevalonate kinase (MK).

In some embodiments, the genetically modified host cell comprises one or more nucleic acids encoding acetoacetyl-CoA thiolase (AtoB), hydroxymethylglutaryl-CoA synthase (HMGS), and hydroxymethylglutaryl-CoA reductase (HMGR) operatively linked to a promoter capable of expression of AtoB, HMGS, and HMGR in the genetically modified host cell. In some embodiments, the genetically modified host cell is capable of converting the MVL into a biofuel.

In some embodiments, the genetically modified host cell is grown in media comprising sucrose. When the genetically modified host cell is grown in media comprising sucrose, the genetically modified host cell is capable of producing MVL at about a yield of about 140 mg/L.

The present invention provides for a composition comprising: (a) the genetically modified host cell of the present invention, and (b) a medium capable of growing or culturing the genetically modified host cell suitable for the genetically modified host cell to produce mevalonate (MVL). In some embodiments, the composition or medium comprises CO, CO₂, H₂, and/or O₂ gas, wherein the concentration of CO, CO₂, H₂, and/or O₂ gas in the medium is saturated or higher when compared to the concentration CO, CO₂, H₂, and/or O₂ gas by merely bubbling air into the medium. In some embodiments, the composition or medium has CO, CO₂, H₂, and/or O₂ gas, or a mixture thereof, or syngas, bubbled through the medium.

In some embodiments, the medium is aerobic. In some embodiments, the genetically modified host cell in the medium has a OD₆₀₀ of equal to or more than about 40, 50, 60, 70, 80, 90, or 100, or is a value within of any two preceding values. In some embodiments, the genetically modified host cell produces MVL with a yield of equal to or more than about 50, 60, 70, 80, 90, 100, 110, 120, 130, 140, or 150, or is a value within of any two preceding values. In some embodiments, bioprocess optimization of the method can result in extremely high cell densities (OD₆₀₀ higher than about 40), and supporting MVL production of about 90 mg/L.

Bench-scale production of mevalonate has been performed and scaled up to about 300 mL using an automated gas bioreactor that allows growth on H₂/CO₂/O₂, as well as CO₂.

The invention can be used for the following: production of a biofuel, such as sesquiterpene or SAF, fragrances, and/or other terpene products from syngas and/or one or more industrial off-gases. Bioprocess optimization is useful for other bioproduction schemes, such as the production of fatty alcohols.

In some embodiments, the host cell is aerobic host. In some embodiments, the method comprises growing or culturing the host cell under aerobic condition. The aerobic CBB cycle produces 3-fold more free energy than the anaerobic WL pathway, which allows aerobic systems to synthesize complex molecules such as sesquiterpenes (up to C15), whereas anaerobic hosts are limited to forming C2-C4 molecules such as ethanol.

The present invention provides for a method for producing a mevalonate (MVL), or a biofuel produced using MVL as a precursor, comprising: (a) providing a medium comprising a genetically modified host cell in a vessel; (b) introducing CO, CO₂, H₂, and/or O₂ gas, or a mixture thereof, or syngas, into the medium; (c) optionally recycling any unused gas introduced in step (b); (d) optionally removing or separating the MVL or biofuel produced from the medium; and (e) optionally removing any biomass generated from the growth of the genetically modified host cell.

In some embodiments, the vessel is a reactor vessel, or cell growth or production tank. In some embodiments, the H₂, and/or O₂ is produced by electrolyzing water using a water electrolyzer.

In some embodiments, the method or system comprises the use of microbial electrochemical fluidized bed reactors (ME-FBR) to convert CO₂ into chemicals with direct and indirect electron transfer approaches. Utilizing fluidized bed electrodes enhances gas-liquid mass transport and electron transfer, facilitating the efficient coupling of electrochemical and biological conversions. In some embodiments, commercial electrocatalysts (Sn, In, and Bi) are used for the fluidized bed cathode. These catalysts enable the reduction of CO₂ to formic acid under highly selective and efficient conditions. These materials are biocompatible with a model microorganism (such as Cupriavidus necator) capable of utilizing formic acid. In some embodiments, these microorganisms can thrive in the presence of suspended particles of these electrocatalysts, adapting rapidly to the environment. In some embodiments, the method or system comprises using ME-FBR comprising fluidized particles (In, Sn, and Bi) for the indirect conversion of CO₂ to formate, and subsequent biological upgrading by C. necator. In some embodiments, for the direct approach, fluidized particles (conductive glassy carbon and activated carbon) are integrated for the conversion of CO₂ to diverse chemical products with the host cell, such as Clostridium ljungdahlii and/or mixed consortia. The results indicate that microbial electrochemical fluidized bed reactors offer innovative approaches to efficiently couple CO₂ electrolysis with biological C1 upgrading.

The present invention provides for methods, systems, host cells, and compositions described herein.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. Plasmid for mevalonate (MVL) production, construct for chromosomal insertion with novel SCRAGE genome engineering method, and mevalonate production profiles during heterotrophic growth for different constructs, grown in minimal media plus 1% sucrose, MVL measured by HPLC. Construct for chromosomal insertion with novel SCRAGE genome engineering method. Mevalonate production profiles during heterotrophic growth for different constructs, grown in minimal media plus 1% sucrose, MVL measured by HPLC.

FIG. 1B. 28 putatively constitutive promoters from DSM1084 were fused to sfgfp, integrated into the chromosome of Hydrogenophaga pseudoflava DSM1034 using SAGE, and tested for resulting fluorescence (FIG. 1). We have identified different promoters with variable strength, including strong promoters.

FIG. 2A. Induction curves for different concentrations of m-toluic acid.

FIG. 2B. Pathway design and prototyping for mevalonate production,

FIG. 3. Heterotrophic mevalonate titers from H. pseudoflava grown on 2% sucrose in minimal media.

FIG. 4. (A) Comparative growth in gas fermentation reactors of H. pseudoflava 1084 W, 1034 W and 1034 NADH.da (MVA pathway). (B) Mevalonate production in a gas reactor.

FIG. 5. (A) Mevalonate production under heterotrophic growth conditions in a batch bioreactor. (B) Cellular growth. (C) Sucrose consumption.

FIG. 6. MVA consumption under heterotrophic conditions. (A) MVA consumption DSM 1034. (B) 1034 O.D.

FIG. 7. MVA consumption under autotrophic conditions. (A) MVA consumption DSM 1034. (B) 1034 O.D.

FIG. 8. (A) Operational expenditures are dominated by energy costs in all scenarios while H₂ costs were more significant when not recycling the CO₂. (B) In contrast, electrolyzer cost was the dominant capital expenditure, while amine scrubbers were also a significant cost when recycling CO₂.

FIG. 9A. Process diagram for CO₂ electrolysis coupled to aerobic fermentation for sesquiterpene production. E=electrical; T=thermal.

FIG. 9B. Process diagram for CO₂ electrolysis coupled to aerobic fermentation for sustainable aviation fuel (SAF) production. E=electrical; T=thermal.

FIG. 10. 23 promoters tested for response to limited nitrogen conditions in DSM1034 by using them to express T7 RNA Polymerase, with a T7-responsive promoter driving sfgfp expression. The promoters were chosen from proteomics data on DSM1084 grown on limited and excess nitrogen concentrations.

FIG. 11. Autotrophic growth of H. pseudoflava is enhanced in the presence of 5% CO. Cells were precultured in low salt LB media overnight, were washed three times and were inoculated into 5 mL of minimal media without a carbon source in 150 mL flasks. Flasks were sealed and vacuumed. Gases were added at indicated quantities and were placed in 30° C. at 200 rpm.

FIG. 12. Colorimetric results from promoter screen.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

### Host Cell

In some embodiments, the host cell is any prokaryotic or eukaryotic cell, with any genetic modifications, taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

In some embodiments, the host cell is any organism described herein.

Generally, although not necessarily, the host cell is a yeast or a bacterium. In some embodiments, the host cell is Rhodosporidium toruloides or Pseudomonas putida. In some embodiments, the host cell is a Gram-negative bacterium. In some embodiments, the host cell is capable of aerobic growth.

In some embodiments, the host cell is of the phylum Pseudomonadota. In some embodiments, the host cell is of the class Betaproteobacteria. In some embodiments, the host cell is of the order Burkholderiales. In some embodiments, the host cell is of the family Comamonadaceae. In some embodiments, the host cell is of the genus Hydrogenophaga. In some embodiments, the host cell is of the phylum Proteobactera. In some embodiments, the host cell is of the class Gammaproteobacteria. In some embodiments, the host cell is of the order Enterobacteriales. In some embodiments, the host cell is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes. Suitable eukaryotic host cells include, but are not limited to, fungal cells. Suitable fungal cells are yeast cells, such as yeast cells of the Saccharomyces genus.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments the host cell is a bacterium. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, or B. pumilus.

### Biofuel

In some embodiments, the biofuel produced is any biofuel which can be produced using MVL as a precursor, described produced in a cell taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

### Example 1

Engineering Hydrogenophaga pseudoflava for the Bioconversion of Gas Substrates to Mevalonate

Hydrogenophaga pseudoflava, an aerobic betaproteobacterium that is known for its ability to oxidize CO. This host has been engineered only once previously (Grenz et. al.) to grow on CO to low biomass accumulation levels (<0.1 DCW/L) while making dilute titers of a terpene, alpha bisabolene (about 75 μg/L). Here, we further developed the genetic toolbox of this host by developing high-efficiency electroporation techniques, identifying stable plasmids, enabling chromosomal integration techniques via allelic replacement and Serine recombinase Assisted Genome Engineering (SAGE), and implementing a metabolic route to biomanufacture mevalonate, a platform chemical that used to produce terpenes, a diverse class of compounds comprising >50,000 unique chemicals that can address markets in the fuels, fragrances, flavors, and pharmaceutical industries. Further, mevalonate can be polymerized to produce flexible, biodegradable, plastic-like polymers.

### Materials and Methods

### Bacterial Strain and Culture Conditions

For routine cultivation and strain engineering of Hydrogenophaga pseudoflava DSM1034, a low-salt modified LB (mLB) media containing 10 g/L Tryptone, 5 g/L Yeast extract, 5 g/L NaCl & trace salts 1.05 M Nitrilotriacetic acid, 0.59 M MgSO₄*7H₂O, 0.91 M CaCl₂*2H₂O, 0.04 M FeSO₄*7H₂O was used and pH of the medium was maintained using NaOH (10% v/v). When antibiotic selection was necessary, kanamycin (25 μg mL⁻¹) or chloramphenicol (10 μg mL⁻¹) was supplemented. Cupriavidus Minimal Media (CMM) was used for all bioproduction experiments (1M NaH₂PO₄, 0.5M Na₂HPO₄, 0.5M K₂SO₄, 1M NaOH, 3.42 mM MgSO₄, 0.42 mM CaCl₂, trace salts, 10% (w/v) NH₄Cl) (Panich et al., 2024) and 2% sucrose was added as a carbon source for heterotrophic batch bioproduction experiments. The samples were induced using 0.5 mM m-toluic acid at indicated time points. For autotrophic cultivation, gas mixtures were used consisting of 62% H2, 10% CO2, 10% O2 purchased from Linde Inc. (Emeryville, CA, USA), Optical Density (O.D.) was measured at 600 nm using Molecular Devices® SpectraMax M2 Spectrophotometer using a cuvette path length of 1 cm,

### Plasmid Design

The pXylsRFPt plasmid containing the broad-host BBR1 medium-copy origin was routinely used to express heterologous genes (Bi et al. 2013). The Mevalonate (MVA) synthesizing gene cassette had AtoB (AcetylCoA-Acetyltransferases) and HMGS (hydroxymethylglutaryl-CoA synthase) from S. cerevisiae. HMGR (3-Hydroxy-3-Methylglutaryl-CoA Reductase) is a key rate limiting enzyme in the MVA synthesis, To test the preference of reducing equivalent supply for H. pseudoflava strain, two different HMGR genes were amplified separately from NADH dependent HMGR (NADH.da) from Delftia acidovorans and NADPH dependent (NADPH.sa) from Staphylococcus aureus using JBx_001376, JBx_001376 respectively (webpage for: public-registry.jbei.org). The fragments were cloned via Gibson assembly. The correct sequence of the whole plasmid pMVA.da and pMVA.sa was determined by plasmid sequencing and PCR.

### Genetic Manipulation and Strain Storage

The cloning fragments were amplified with Phusion® High-Fidelity DNA Polymerase (NEB, Ipswich, MA, USA) or Q5® High-Fidelity DNA Polymerase (NEB, Ipswich, MA, USA), The PCR products were purified using the QIAquick Gel Extraction Kit (Qiagen, Hilden, Germany) and were digested with Dpn1 (NEB, Ipswich, MA, USA). Ligation and assembly of DNA fragments were performed with NEBuilder® HiFi DNA Assembly Master Mix (NEB, Ipswich, MA, USA) according to the manufacturer's protocol. The transformation was carried out via heat shock (CaCl₂) method) into E. coli S17-1 cells. Plasmid-carrying E. coli strains were grown in the presence of 50 ng mL-1 kanamycin. QIAprep Spin Miniprep Kit (Qiagen, Hilden, Germany) separated plasmids from E. coli strains grown overnight according to the manufacturer's protocol. Whole plasmid sequencing was performed by Primordium Labs (Arcadia, CA).

After confirmation of the correct sequence the plasmids were transformed into chemically competent E. coli. AG2074 strain for mimicking H. pseudoflava methylation pattern upon MVA plasmids (Riley and Guss et. al 2021). A modified CaCl₂ method was used to make DSM 1034 competent cells to facilitate transformation via electroporation in H. pseudoflava as bacterial conjugation via E. coli 2074 (auxotrophic for Diaminopimelic acid) strains wasn't successful in our experience. A 5 mL culture of DSM 1034 was grown overnight at 37° C. at 225-250 rpm. 1 ml of this overnight culture was used to inoculate 50 mL of mLB shake flask culture and incubated at 37º° C. until it reached desired O.D. of 0.8. to produce highest competency cells. Cells were pelleted by centrifugation at 6500×g for 10 minutes at 4° C. and washed three times by resuspending in 5 mL cold 10% glycerol. The washed cell pellet was resuspended in 1/50 volume of original culture (1 ml for 50 ml culture) in 10% glycerol and used for replicating plasmids or non-replicating plasmids. These cells can also be stored at −80° C.

For transformation via electroporation in DSM 1034, 100 ng-1 μg of methylated plasmid DNA was added to 50-75 μL of freshly prepared DSM 1034 competent cells. Electroporation was performed at 1.6 kV; 25 uF, 200 ohms using 0.1 cm cuvette and transferred to 950 μL of mLB in a 1.5 mL eppendorf tube and incubated at 37° C. for 1-2 h at 225 rpm. 100 μl of the transformed culture was plated on mLB agar plates containing 25 μg mL⁻¹ kanamycin and incubated at 30° C. until H. pseudoflava colonies were observed. For strain storage, cells were inoculated into mLB medium and CMM medium with appropriate antibiotics and were incubated overnight. Cells were pelleted to concentrate the biomass and were resuspended in 15% (v/v) glycerol, placed in cryogenic tubes, and stored at −80° C.

### Mevalonate Production in 2 L Bioreactor

The autotrophic experiments were performed in liquid batch mode using bioXplorer® 400P (HEL Ltd., Hempstead, United Kingdom) bioreactor with a working volume of 400 mL. The WinIso® software was used for online monitoring and control systems of the reactors under a continuous gas supply. The four bioreactors were run in parallel. The bioreactors had the pressure, temperature, dissolved oxygen (DO), agitation, and pH controllers. The gas supply was provided using a micro sparger located at the vessel's base. Mass Flow Controllers regulated the gas flow rates. The bioreactor vessels were cleaned and autoclaved before adding the CMM medium. The medium was filtered through a 0.2 uM filter and added to the assembled bioreactor vessels under sterile conditions. A gaseous mixture of 85% H₂, 8% O₂, and 10% CO₂ was supplied at 1 bar at 30° C. The H. pseudoflava heterotrophic growth culture was injected into the vessel to maintain an initial O.D. of 0.1. The agitation speed was set at 400 rpm, and pH was adjusted at 6.5 using filtered 3 M NaOH. The NaOH used in the medium also helped to replenish the nitrogen in the medium.

The Heterotrophic growth experiment was performed using Thermo Scientific (Add specifications here). Bioreactors suitable for sucrose based fermentation under fed batch conditions at 0.5 vvm flow rate at 400-500 rpm speed under a dO cascade of 20% and pH was maintained at 6.9. The strains were grown under 10 g/L sucrose in CMM media at 30° C. Inducer (m-toluic acid, 0.5 mM) was added as specified for each experiment.

### Quantification of Mevalonate

The mevalonate production was quantified by HPLC (Ultimate 3000, Thermo Fisher Scientific, Waltham, MA) equipped with an Aminex HPX-87H column (300 mm×7.8 mm, Bio-Rad, Hercules, CA) and Refractive Index detector using standards with known concentrations. The mobile phase containing 4 mM of H₂SO₄ in HPLC grade water was used at a flow rate of 0.4 mL min⁻¹ at a column oven temperature set at 40° C. The samples were collected after every 12 h and 24 h for heterotrophic and Autotrophic growth cultivations. The samples were centrifuged at 13000 rpm for 10 minutes to separate cell biomass from the supernatant. The supernatant was collected and filtered through a 0.2 μm filter to ensure the removal of any cell debris. The samples were injected into HPLC and analyzed against a standard mevalonate curve (1.25 g/L-10 g/L) prepared by dissolving the mevalonate standard in water. 1 mM DL-mevalonate was prepared by mixing 1 volume of 2 M DL-mevalonolactone (Sigma Aldrich) with 1.02 volumes of 2 M KOH and incubating at 37° C. for 30 min. The peak formation at 17.4 RT confirmed the presence of mevalonate in our samples.

### Mevalonate Consumption

Wild-type H. pseudoflava strains DSM 1034 were evaluated for mevalonate consumption under both heterotrophic and autotrophic growth conditions to investigate this further. The heterotrophic growth experiment used a modified LB (mLB) medium. The cultures were maintained at 30° C. with 200-rpm shaking overnight with initial OD₆₀₀ at 0.01 in 5 mL mLB medium. Two different MVA conc. 900 mg/L & 1800 mg/L were used and a control (Ctrl) without mevalonate was also used as a reference. The cultures were incubated for 48 hours in rotary shakers.

### Results & Discussion

We sought to expand the use of this host by developing stable vectors, inducible and constitutive promoters, genome integration methods, and defined pathways to further develop the MVA pathway for terpene synthesis in this host. To increase electroporation efficiency, methylome analysis of H. pseudoflava strains DSM 1034 and DSM 1084 was carried out as previously described (cite), Upon identification of relevant restriction enzymes for each H. pseudoflava strain, E. coli cloning strains defective for endogenous restriction systems were modified to heterologously express the restriction system from each H. pseudoflava strain on the chromosome under the P_{araBAD} system. We report a high efficiency of electroporation for each H. pseudoflava strain when plasmids were purified from their cognate E. coli strain expressing the proper restriction enzyme for each strain.

Initial experiments to develop a genetic toolbox for this host involved screening H. pseudoflava strains DSM 1034 and DSM 1084 for inducible promoters by visually examining REP expression on plates. We found that only one of the promoters (pXyls/PM) consistently worked in both strains. In contrast with previous studies (Grenz et. al.), IPTG-inducible systems failed in our hands.

### Design Rationale

The H. pseudoflava strain possesses a MEP pathway that enables isoprenoid production via its natural metabolism. However, the NADPH consumption via MEP metabolism is not energy efficient and creates metabolic burden. While MVA pathway generates similar metabolites IPP and DMAPP, the MEP pathway consumes fewer lesser NADPH, thus making the bioproduction energy more efficient.

### Promoter Screening

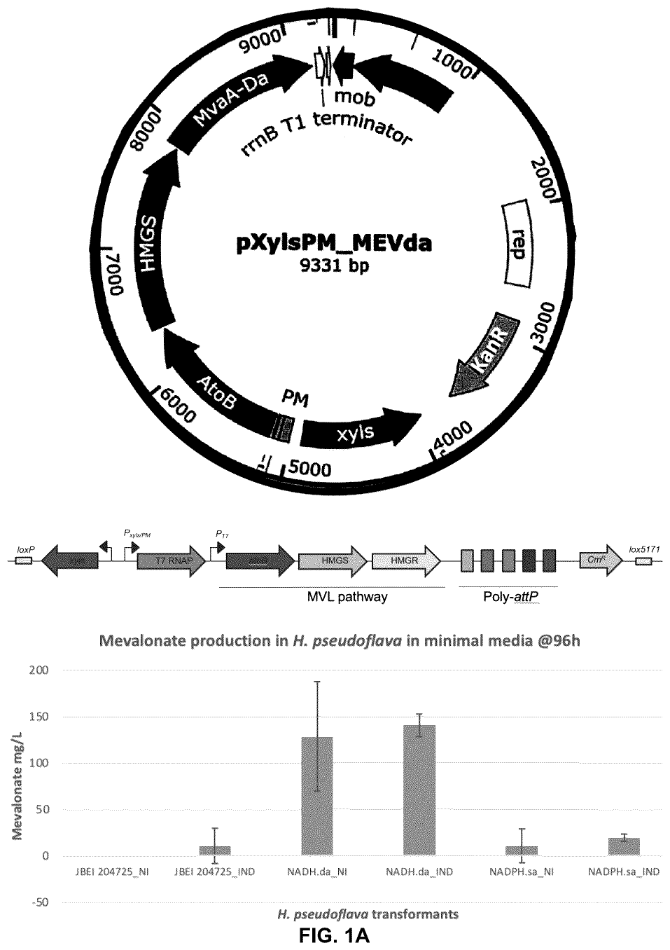

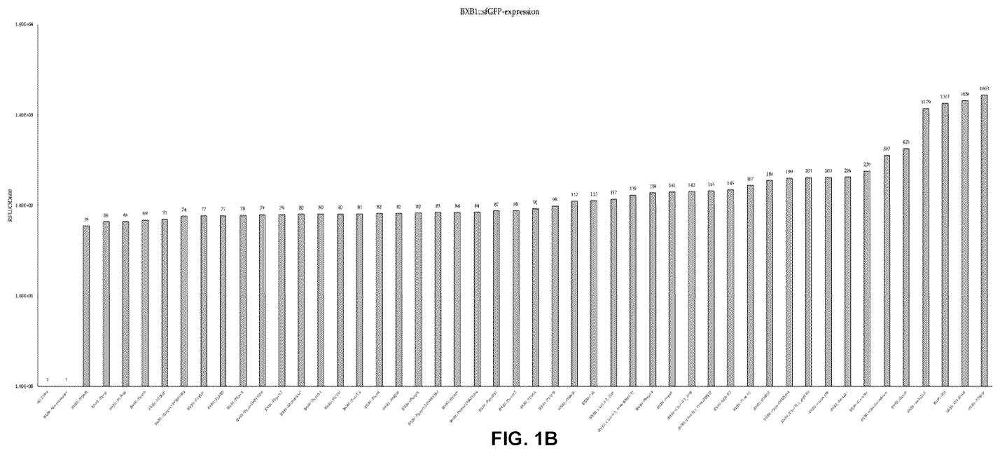

28 putatively constitutive promoters from DSM1084 were fused to sfgfp, integrated into the chromosome of Hydrogenophaga pseudoflava DSM1034 using SAGE, and tested for resulting fluorescence (FIG. 1B), We have identified different promoters with variable strength, including strong promoters.

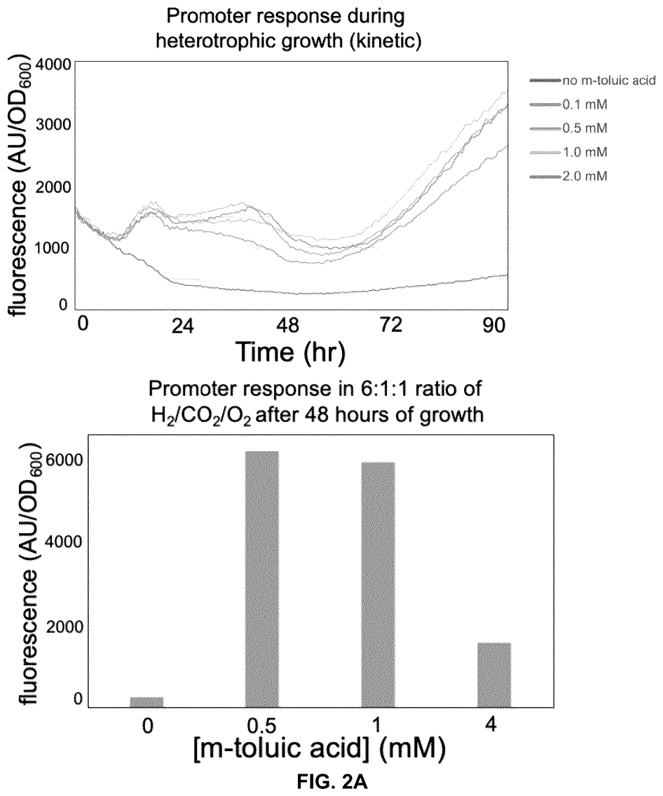

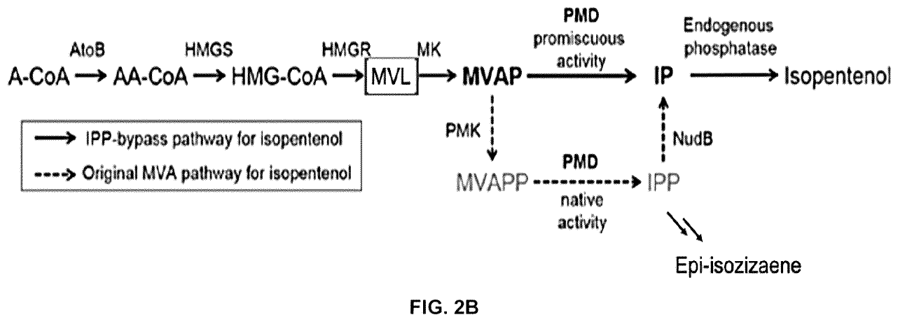

Though we observed a nearly ten-fold dynamic range in constitutive promoter activity, no promoter was particularly strong in this host. Further bioproduction requires temporal control over pathway expression. Therefore, we screened six common inducible expression systems using an RFP reporter, as available in the JBEI registry, including pXyls/PM, pLacIUV5, pAraBAD, pTetA, pCM, and an arabinose-inducible T7 system. We only observed visible red colonies in the clones containing the pXyls/PM promoter under 1 mM m-toluic acid induction. Further experimentation revealed that the pXyls/PM promoter is active under both heterotrophic (sucrose) growth and autotrophic growth conditions (FIG. 2A).

### Strain Engineering—Plasmid Integration

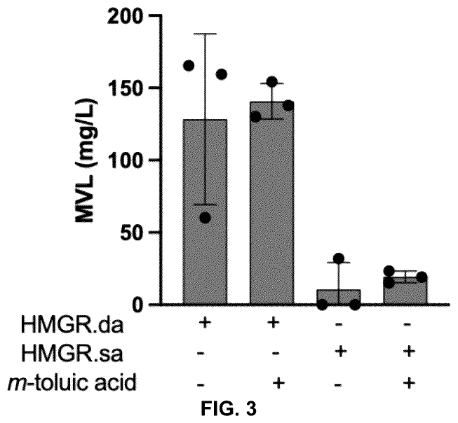

We aimed to produce the terpene precursor molecule and platform chemical, mevalonate (MVA) from organic carbon sources as well as waste gaseous carbon streams CO₂ and CO. Mevalonate (MVA) is an intermediate in the IPP bypass pathway for isoprenol biosynthesis and the epi-isozone-producing pathway. H. pseudoflava as a functional chassis for bioproduction. To this end, we designed a three-gene pathway for the production of MVA using AtoB, HMG synthase from, and HMG reductase. HMG reductase is known to be the rate-limiting enzyme in this pathway, which prompted us to test the best-in-class enzymes with dependencies on NADH and NADPH, hypothesizing that the NADH-dependent enzyme from D. acidovorans will have higher productivities of mevalonate under autotrophic conditions, considering the CBB cycle consumes a large proportion of the cell's NADPH supply. We were surprised to find that the NADH-dependent pathway produced relatively higher titers of mevalonate even when cells were grown on sucrose as a sole carbon source (FIG. 3). We note that MVA concentration values were extrapolated because the standard curve was only performed for 1.25 g/L MVA to 10 g/L MVA, this resolution will improve as we refine our analytical methods for MVA detection. Inducer (0.5 mM m-toluic acid) was added in mid-log phase eight hours after inoculation.

### Evaluation of Mevalonate Production in DSM 1034 Under Autotrophic Growth Conditions

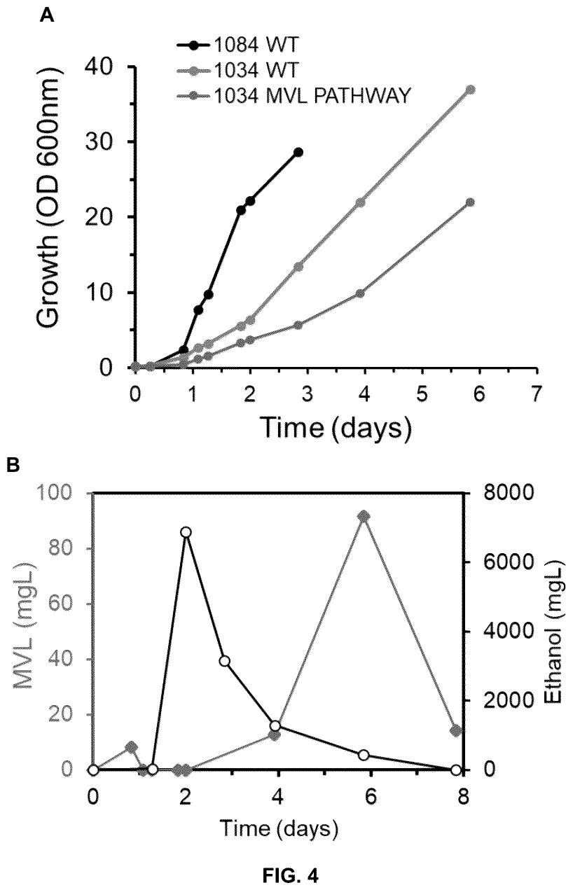

We compared and tested the growth of wild-type H. pseudoflava with our best-performing strain for mevalonate production in mixtures of H₂, O₂ and CO₂. The growth of 1034 required a two-step adaptation to autotrophic conditions in bioreactors. FIG. 4, Panel A shows the growth of both the strains after the first acclimation bioreactor step under autotrophic conditions. Slower growth was observed for the heterologous strain compared to wild-type, with doubling times of 5.5 h for WT and 4 h for 1034 the engineered strain, giving biomass concentrations of 11 g-dw/L and 17 g-dw/L, respectively. The detrimental growth impact for the engineered strain is expected, due to the metabolic burden of heterologous product expression. Maximum titers of MVA in autotrophic conditions were 92 mg/L, spiking at day 6 of the experiment (FIG. 4, Panel B). However, an observed decrease in MVA titer at day 8 indicated plausible mevalonate consumption by the strain.

### Evaluation of Mevalonate Consumption in DSM 1034 Under Heterotrophic Growth Conditions

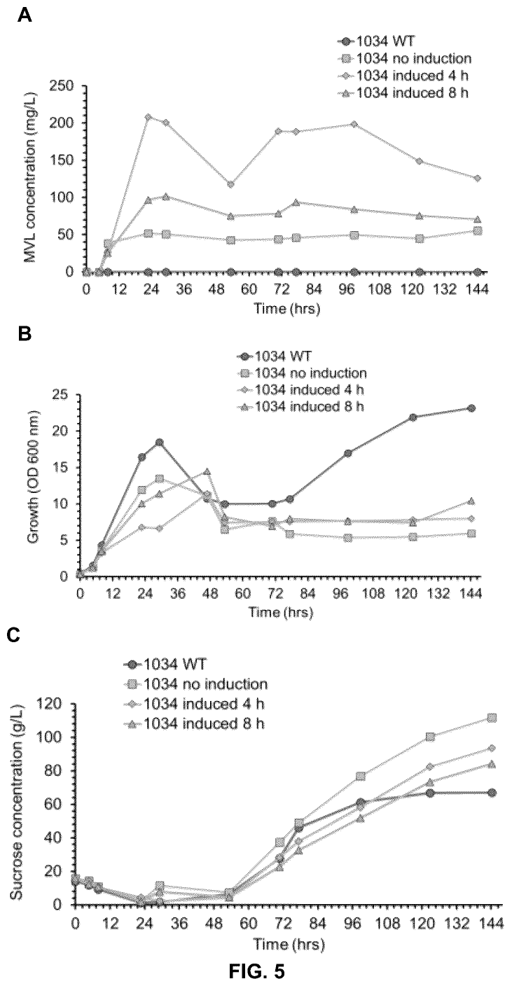

The fermentation results suggest that we could achieve relatively high titers of mevalonate in batch heterotrophic bioreactor experiments. To test this hypothesis, we performed a bioproduction experiment in fed-batch conditions, examining the effect of two different time points for MVA cassette induction (4 h and 8 h). In these experiments, the engineered strain showed maximum mevalonate production of 220 mg/L, also characterized by a slight decrease in titer between 24-50 h and after 96 h, most pronounced when the MVA cassette was induced at 4 h (FIG. 5, Panel A).

### Mevalonate Consumption

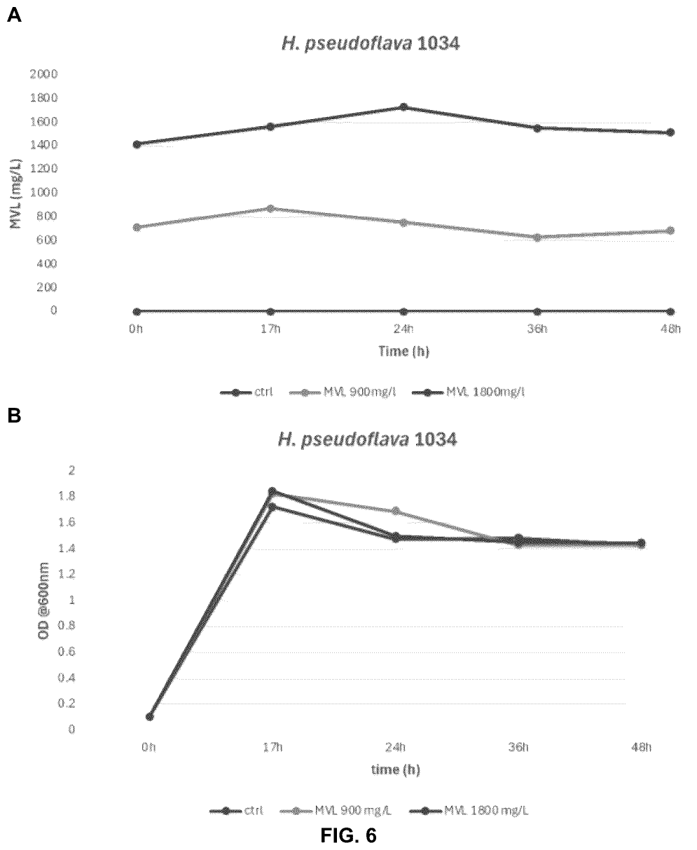

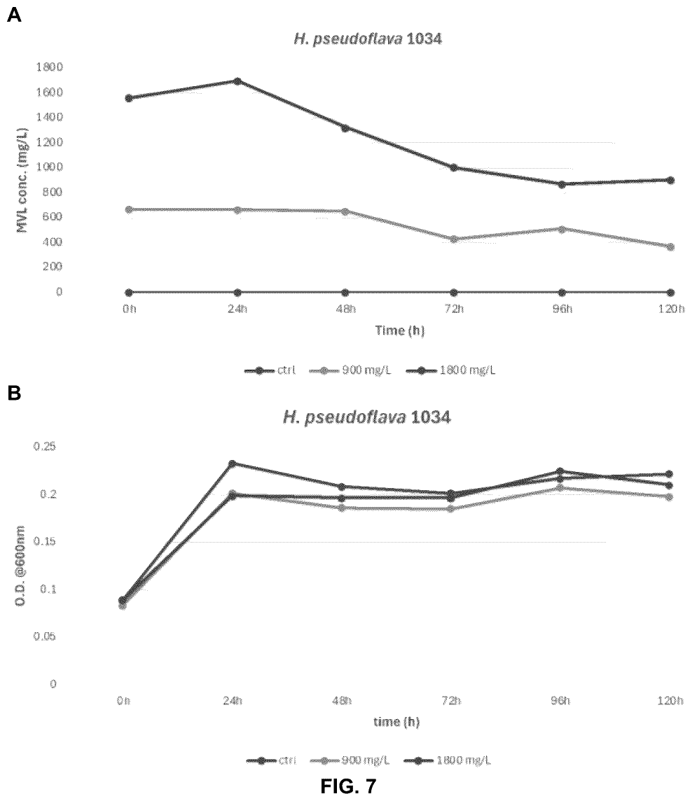

In our initial experiments, no significant MVA consumption was observed in H. pseudoflava strains 1034 under heterotrophic conditions (FIG. 6). The highest cell density observed after 48 hours of incubation were 1.8 for both strains, which is comparable to control conditions. However, when the results were compared to MVA consumption under autotrophic conditions, the addition of gases promoted the consumption of mevalonate wild-type strain 1034 (FIG. 7). The MVA signal ranges from 667.69 mg/L to 367 mg/L & 1557.98 mg/L to 903.57 mg/L in 1034 cultures in 120 h (FIG. 7). The drop in mevalonate under autotrophic conditions indicates low titers reported at the end of fermentation in the engineered strain. These results suggested a relatively higher titer could be achieved if we knockout mevalonate-consuming pathways. However, we were unable to identify such pathways, and proteomics experiments were inconclusive.

Techno Economic Analysis of Electrolysis-Coupled Bisabolene Production Using H. psuedoflava

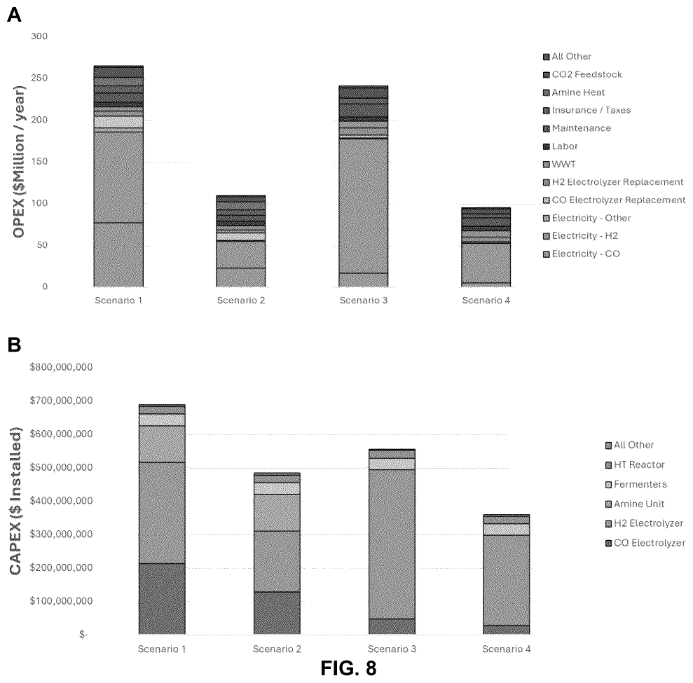

Mevalonate is an intermediate for terpene synthesis. Bisabolene is a terpene chemical that has been widely regarded as a potential SAF candidate in recent years (cite). We modeled sesquiterpene production with and without CO2 recycling (enabled by an amine scrubber) under two economic scenarios (Scenario 1: Electricity Price: $0.068/kWh, Electrolyzer CAPEX: $1,330/KW, CO₂ Cost: $40/ton; Scenario 2: Electricity Price: $0.02/kWh, Electrolyzer CAPEX: $800/KW, CO₂ Cost; $20/ton). We found with CO2 recycling, the minimum selling price (MSP) was $18.07/gallon neat SAF under economic scenario 1 and $9.54/gallon SAF under economic scenario 2. Interestingly, MSP was reduced when CO2 was not recycled, offering an MSP of $15.85/gallon SAF in economic scenario 1 and $7.64/gallon SAF in economic scenario 2. We found that factoring the value of accumulated biomass ($1.50/kg) further reduced the MSP of SAF in each scenario by $2.50. In summary, not recycling CO2 was advantageous and assuming the most favorable economic conditions would allow for an MSP of $5.14/gallon SAF. Operational expenditures were dominated by energy costs in all scenarios while H2 costs were more significant when not recycling the CO2 (FIG. 8).

In contrast, electrolyzer cost was the dominant capital expenditure, while amine scrubbers were also a significant cost when recycling CO2 (FIG. 8), Therefore, progress must be made to drive down unit costs for electrolyzers, increasing their durability, or both.

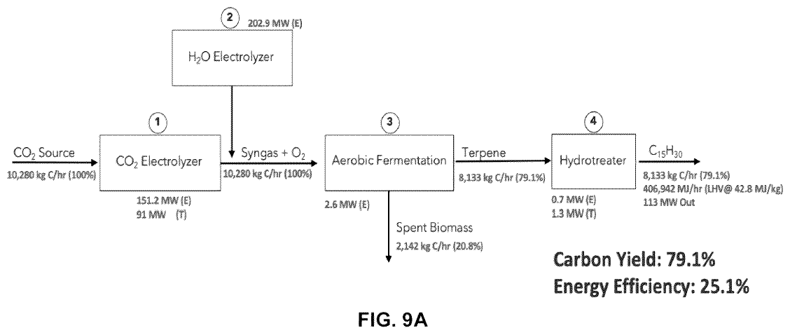

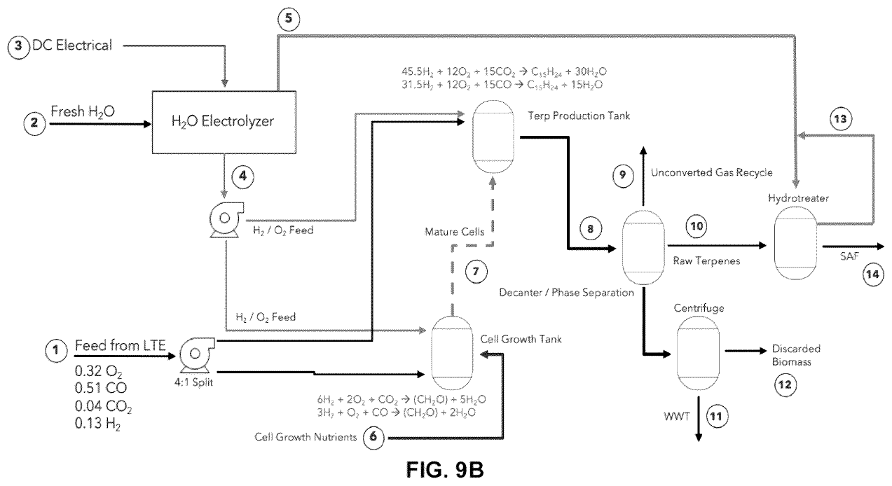

FIGS. 9A and 9B show the process diagram for CO₂ electrolysis coupled to aerobic fermentation for sesquiterpene or SAF production. Microbial growth was modeled using a simplified 8 e−/Ceq reaction, 3H₂+O₂+CO-> (CH₂O)+2H₂O, while sesquiterpene production was modeled using the simplified formula 31.5H₂+12O₂+15CO->C₁₅H₂₄+15H₂O (93 e−/sesquiterpene).

### Large-Sized Pathway Constructs

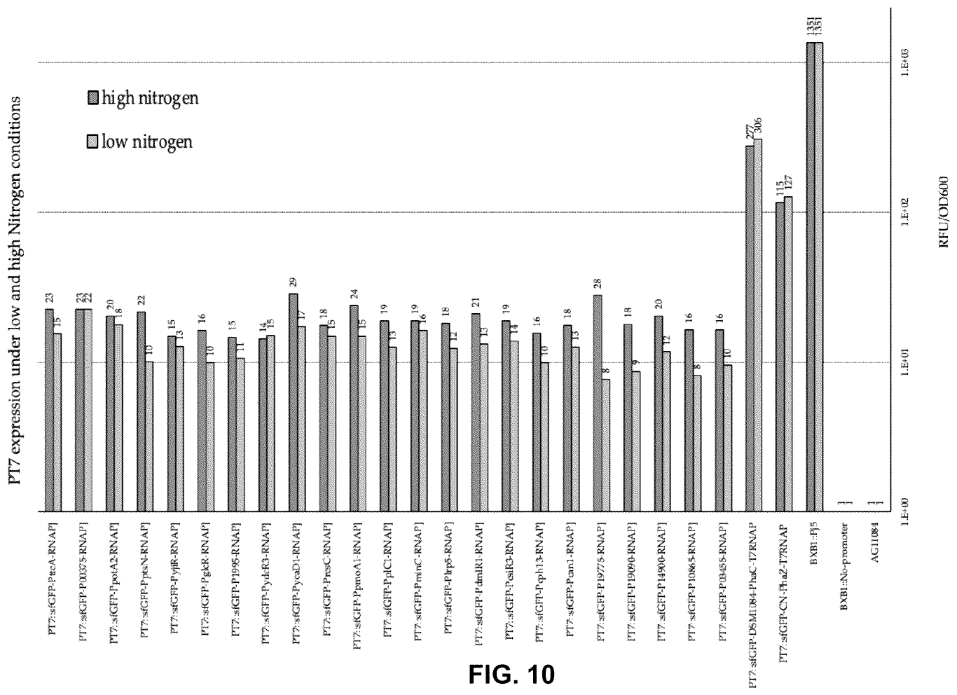

The complete IPP Bypass assembly (15,222 bp) and LacO Plasmid assembly (18,419 bp) were transformed in DSM 1034 H. pseudoflava to express the isoprenol or epi-isozizane pathway. Both electroporation and conjugation were performed as methods of transformation in the host strain DSM 1034. The Plasmids were unstable, and the host strain rejected either the complete plasmid DNA or different gene cassettes of the Plasmid DNA. In an alternative approach, to avoid plasmid DNA rejection and better stability; plasmid DNA was methylated using the strains developed at the Adam Guss lab (Tidwell, A. K., Faust, E., Eckert, C. A., Guss, A. M., & Alexander, W. G. (2024). Discovering methylated DNA motifs in bacterial nanopore sequencing data with MIJAMP. BioRxiv, 2024-08), and a new media composition mLB (modified LB). The new media composition supported the growth of our host strain with higher ODs (6-7) over shorter incubation times (24 h), however, the plasmid stability issue remained the same (FIG. 10), likely due to the large size of constructs encoding our pathways of interest.

T7 RNAP System Fails in H. pseudoflava

We tested 23 promoters that potentially respond to limited nitrogen conditions in DSM1034 by using them to express T7 RNA Polymerase, with a T7-responsive promoter driving sfgfp expression. The promoters were chosen from proteomics data on DSM1084 grown on different nitrogen concentrations (limited and excess N) (FIG. 10). No expression was seen from any of the promoters, potentially suggesting a problem with the T7 RNA Polymerase.

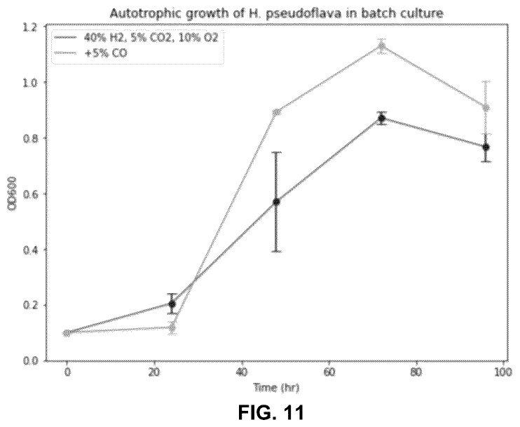

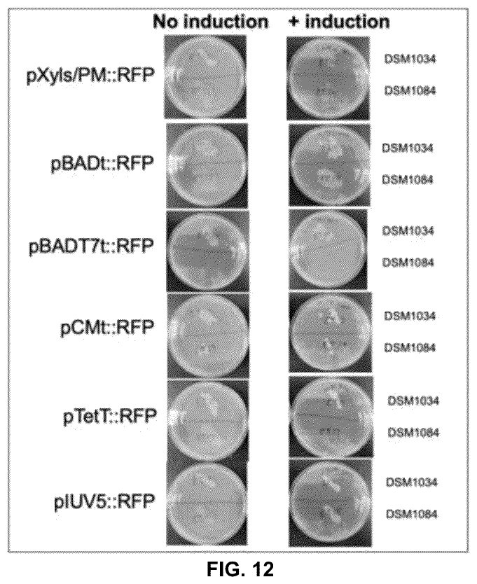

FIG. 11 shows autotrophic growth of H. pseudoflava is enhanced in the presence of 5% CO. Cells were precultured in low salt LB media overnight, were washed three times and were inoculated into 5 mL of minimal media without a carbon source in 150 mL flasks. Flasks were sealed and vacuumed. Gases were added at indicated quantities and were placed in 30° C. at 200 rpm. FIG. 12 shows the colorimetric results from a promoter screen.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A genetically modified host cell capable of converting syngas or a mixture of CO₂/H₂ to produce mevalonate comprising one or more nucleic acids encoding acetoacetyl-CoA thiolase (AtoB), hydroxymethylglutaryl-CoA synthase (HMGS), and hydroxymethylglutaryl-CoA reductase (HMGR) operatively linked to a promoter capable of expression of AtoB, HMGS, and HMGR in the genetically modified host cell.

2. The genetically modified host cell of claim 1, wherein the genetically modified host cell is capable of converting the MVL into a biofuel.

3. The genetically modified host cell of claim 2, wherein biofuel is a sesquiterpene or sustainable aviation fuel (SAF).

4. The genetically modified host cell of claim 1, wherein the genetically modified host cell is a Hydrogenophaga cell.

5. The genetically modified host cell of claim 4, wherein the genetically modified host cell is a Hydrogenophaga pseudoflava.

6. A method for producing a mevalonate (MVL), or a biofuel produced using MVL as a precursor, comprising: (a) providing a medium comprising a genetically modified host cell in a vessel; (b) introducing CO, CO₂, H₂, and/or O₂ gas, or a mixture thereof, or syngas, into the medium; (c) optionally recycling any unused gas introduced in step (b); (d) optionally removing or separating the MVL or biofuel produced from the medium; and (e) optionally removing any biomass generated from the growth of the genetically modified host cell.
